(19) United States
(12) Patent Application Publication
(10) Pub. No.: US 2018/0154040 A1
(43) Pub. Date: Jun. 7, 2018

(54) SCAFFOLD PREPARING METHOD AND SCAFFOLD PREPARED THEREBY

(71) Applicants: Duk Hyun LEE, Daegu (KR); OSSGEN, Daegu (KR)

(72) Inventors: Woo Kie MIN, Daegu (KR); Dong Hoon LEE, Seoul (KR); Hyeon Jun KIM, Busan (KR); Jae Hyuk YANG, Yonginsi, Gyeonggi-do (KR); Jae Hyuk YANG, Seoul (KR); Lih WANG, Busan (KR); Duk Hyun LEE, Daegu (KR)

(21) Appl. No.: 15/578,576

(22) PCT Filed: Oct. 30, 2015
(86) PCT No.: PCT/KR2015/011559

§ 371 Date: Nov. 30, 2017

(30) Foreign Application Priority Data
Jun. 4, 2015 (KR) 10-2015-0078905

(51) Int. Cl. A61L 27/34; A61L 27/12; A61L 27/56; A61L 27/54

(52) U.S. Cl. CPC A61L 27/34; A61L 27/12; A61L 27/56; A61L 27/54; A61L 2300/412; A61L 2430/02; A61L 2300/602; A61L 2300/802; A61L 2420/02; A61L 2420/06; A61L 2300/252

(57) ABSTRACT

The present invention relates to a scaffold preparation method and a scaffold prepared thereby, and more particularly to a method for preparing a scaffold for promoting bone regeneration, the method comprising physically and chemically attaching collagen and SDF-1 to the scaffold, and to a scaffold prepared thereby. According to the present invention, the surface of a scaffold is treated with 3-APTES so that collagen and SDF-1 can be physically and chemically attached to the scaffold, so that the release rate of SDF-1 on the scaffold will not rapidly change even with the passage of time while integrin will recognize SDF-1 of the scaffold as a suitable extracellular matrix, thereby shortening the time required for restoration of bone defects.

### TECHNICAL FIELD

The present invention relates to a scaffold preparing method and a scaffold prepared thereby, and more particularly to a method for preparing a scaffold for promoting bone regeneration, the method comprising physically and chemically attaching collagen and SDF-1 to the scaffold, and to a scaffold prepared thereby.

### BACKGROUND ART

Extracellular substances include an organic solid substance, called extracellular matrix (ECM) based on organic polymers such as proteins and polysaccharides. The ECM serves as a structural support for tissue and to induce cellular adhesion.

When cells adhere to the ECM, intracellular signaling is activated, and fundamental cell functions such as cell morphology, proliferation, cell death and the like are controlled. Thus, a scaffold that performs the role of the extracellular matrix (ECM) is prepared artificially and used not only for bone grafting, but also in the tissue engineering field in which patient's stem cells are attached to the scaffold and transplanted into bone defect sites. In the prior art related to the scaffold, Korean Patent No. 10-1230704 discloses a scaffold comprising calcium phosphate, and Korean Patent No. 10-1436740 discloses a scaffold comprising natural polymers such as gelatin, collagen, chitosan and the like.

Such scaffolds comprise specific signaling molecules which are delivered to stem cells in suitable amounts at suitable timing to control the phenotype of the stem cells. Examples of the signaling molecules in the scaffolds include chemotactic signaling molecules known as chemokines. SDF-1 (stromal derived factor-1) which is a chemotactic signaling molecule is contained in a scaffold and serves to guide stem cells into the scaffold, thereby promoting bone regeneration.

Technologies comprising applying SDF-1 to scaffolds as described above include Korean Patent No. 10-1436740. According to the disclosure of the Korean Patent, a scaffold comprising natural polymers such as gelatin, collagen, chitosan and the like cannot maintain its structure in vivo for a long period of time due to its high biodegradability. In order to overcome this problem, gelatin is mixed with siloxane to provide a gelatin-siloxane hybrid scaffold which is not degraded in vivo and which has high stability and mechanical strength. In addition, SDF-1 is incorporated into the hybrid scaffold so that the hybrid scaffold can slowly release SDF-1 in vivo while maintaining excellent physical and chemical stability in vivo, indicating that the hybrid scaffold can be effectively used for tissue regeneration.

However, the scaffold disclosed in the above-described Korean Patent is a scaffold prepared by mixing the natural polymer gelatin with siloxane and has the following problems. Namely, cellular integrin does not recognize the siloxane as a suitable extracellular matrix, and thus the proliferation and differentiation rates of cells are somewhat slow. Furthermore, no amino group is produced on the surface of the gelatin-siloxane hybrid scaffold, and thus the scaffold does not form a chemical bond with the growth factor SDF-1 by itself, and contains SDF-1 only by physical capillary force. For this reason, when the scaffold is implanted in vivo, SDF-1 is released early before the adhesion of cells to the scaffold, after which the amount of SDF-1 released decreases rapidly with the passage of time. This indicates that the scaffold cannot exhibit a long-lasting cell regeneration effect.

Meanwhile, SDF-1 may also be applied to a calcium phosphate scaffold disclosed in Korean Patent No. 10-1230704 owned by the applicant. However, the calcium phosphate scaffold has the following problem. Namely, the calcium phosphate scaffold has no amino group on the surface thereof, and for this reason, when SDF-1 is applied to the scaffold, SDF-1 can bond to the scaffold only in a physical manner by a capillary force and does not form a chemical bond with the scaffold. Thus, when the scaffold is applied to the human body, SDF-1 in the scaffold is released rapidly, indicating that the scaffold cannot exhibit a cell regeneration effect.

### DISCLOSURE

### Technical Problem

The present invention has been made in order to solve the above-described problems, and it is an object of the present invention to provide a scaffold whose surface has an amino group produced by treating the surface with 3-APTES so that collagen and SDF-1 can be physically and chemically attached to the scaffold, so that the release rate of SDF-1 on the scaffold will not rapidly change even with the passage of time while integrin will recognize SDF-1 of the scaffold as a suitable extracellular matrix, thereby shortening the time required for restoration of bone defects.

### Technical Solution

To achieve the above object, the present invention provides a scaffold preparation method, comprising the steps of: (S1) treating the surface of a scaffold with 3-APTES; (S2) preparing a mixture solution of collagen and SDF-1; and (S3) immersing the calcium phosphate scaffold, surface-treated with 3-APTES in step (S1), in the solution of step (S2).

The scaffold that is used in the method is a calcium phosphate scaffold.

The mixture solution of collagen and SDF-1 in step (S2) is prepared by:

adding collagen to distilled water, and adding and dissolving acetic acid therein to obtain a first solution;

mixing 1-ethyl-3-(3-dimethylaminopropyl)carbodiimide (EDC) and N-hydroxysuccinimide (NHS) with distilled water to obtain a second solution;

mixing the first solution and the second solution with each other at a ratio of 1:1 to obtain a mixture, and maintaining the mixture for 6 hours or more; and

adding SDF-1 to the mixture when the mixture reaches a pH of 5.5-4.7, and completely dissolving the SDF-1.

### Advantageous Effects

According to the present invention configured as described above, SDF-1 and the carboxyl group of collagen are simultaneously attached to the scaffold through an amino group produced by treating the surface of the scaffold with 3-APTES. Thus, when the scaffold is implanted into the human body, physically attached SDF-1 can be released at an early stage to promote the migration of stem cells to the scaffold, and chemically attached SDF-1 can serve as an ECM which is involved in continuous migration of cells to the scaffold. In addition, collagen in the scaffold enables cellular integrin to recognize SDF-1 as a suitable ECM, thereby increasing the adhesion and growth rate of cells on the scaffold.

Thus, the proliferation and differentiation rates of cells on the scaffold increases, leading to an increase in bone regeneration rate, which greatly contributes to reducing the time required for restoration of bone defects.

In addition, according to the present invention, a scaffold showing high bone regeneration rate can be prepared using a conventional scaffold.

### DESCRIPTION OF DRAWINGS

FIG. 1 is an SEM image of a BCP scaffold.

FIG. 2 is a schematic view showing a process in which the surface of a scaffold is treated according to the present invention and collagen and SDF-1 are attached to the scaffold.

FIG. 3 shows the results of XPS analysis of the surface of a scaffold according to the present invention.

### MODE FOR INVENTION

Hereinafter, embodiments of the present invention will be described with reference to the accompanying drawings.

Recent studies on stem cells have been conducted mainly on adult stem cells, particularly mesenchymal stem cells and hematopoietic stem cells. Mesenchymal stem cells pose no ethical problems and cause no immune rejection, and thus receive an attention as a useful tool for cell therapy in the future.

The term “stem cell mobilization” means that the mesenchymal stem cells in the bone marrow niche migrate from the bone marrow to the systemic circulation in response to a specific signal from distant damaged tissue.

Furthermore, the term “homing” means that mobilized stem cells are captured by the blood vessels of their target tissue and transmigrate into the endothelium. Cell surface proteins known to be associated with homing include SDF-1a (stromal cell-derive factor-1a) and its receptor (CXCR-4; CXC chemokine receptor-4). In a tissue that was damaged or needs to be regenerated, the concentration gradient of SDF-1a is formed so that stem cells will migrate to their target tissue through CXCR4 expressed on the surface thereof, and rolling and adhesion of the stem cells will occur.

In conclusion, SDF-1 enables cells to migrate to a desired site through homing of the cells. Thus, SDF-1 enables stem cells to migrate to a wound, making it possible to rapidly repair the wound.

Meanwhile, collagen is one of the most abundant proteins in human body, and it is known that type I collagen is also present in natural bone. When collagen is used, integrin recognizes the collagen as a suitable extracellular matrix to facilitate cell adhesion.

As the cell adhesion time is shortened as described above, rapid proliferation and differentiation of the cells becomes possible, which is important in rapid bone regeneration.

According to the present invention, when the scaffold comprising the SDF-1 and collagen effective for bone regeneration is implanted into bone defects, it can greatly contribute to reducing the restoration time of the bone defects by increasing bone regeneration rate.

Hereinafter, examples of the present invention will be described in further detail with reference to the accompanying drawings. However, these examples are for illustrative purposes and are not intended to limit the scope of the present invention.

(1) Preparation of Scaffold

It is known that a calcium phosphate scaffold is a component of human natural bone, serves as an extracellular matrix (ECM), shows excellent cell adhesion and osteoconductivity, and has high biocompatibility, bioactivity and cellular conductivity. The calcium phosphate scaffold has higher bioactivity and mechanical strength than a conventional gelatin-siloxane hybrid scaffold.

Particularly, in the present invention, a BCP-containing calcium phosphate scaffold (hereinafter referred to as “calcium phosphate scaffold”) is prepared and used. This is because BCP (biphasic tricalcium phosphate) comprises hydroxyapatite (HAp, Ca₁₀(PO₄)₆(OH)₂), which is most similar to natural bone, together with β-tricalcium phosphate (β-TCP, Ca₃(PO₄)₂). HAp forms a strong chemical bond with human bone and has relatively high strength, and β-TCP has excellent biodegradability. Thus, BCP can further increase the mechanical strength and biodegradability of the scaffold.

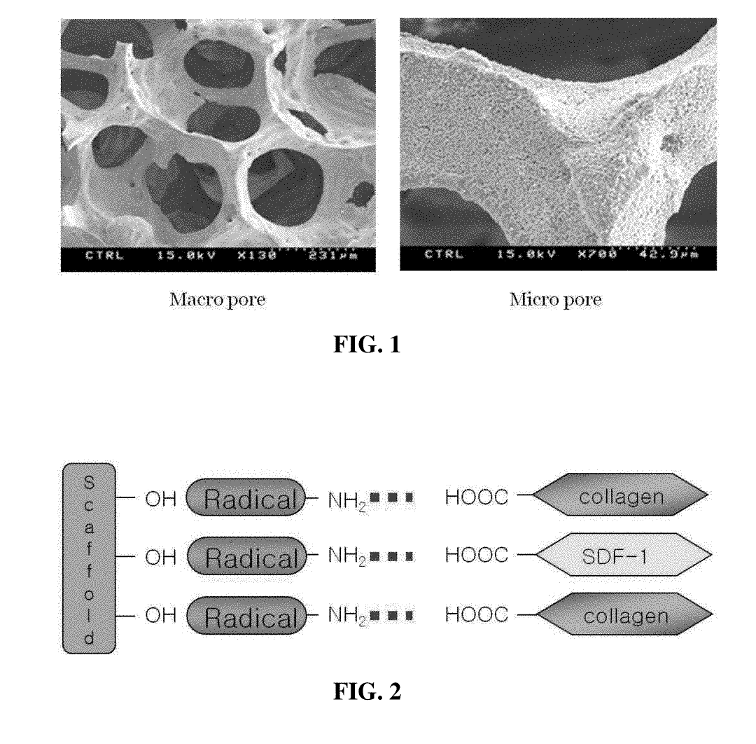

This scaffold is already disclosed in the registered patent owned by the applicant, and thus a method for preparing the same is omitted herein. As shown in FIG. 1, the scaffold includes macropores having a size of 200 to 400 μm, and micro open pores having a size of 0.5 to 5 μm.

(2) Surface Treatment with 3-Aminopropyltriethoxysilane (3-APTES)

In a pretreatment process before coating SDF-1 and collagen on the porous calcium phosphate prepared in (1) above, the surface of the scaffold is treated with 3-APTES.

The porous calcium phosphate scaffold described in (1) has no amino group on the surface thereof, and for this reason, when the surface of the scaffold is not treated, it cannot form a chemical bond with collagen and SDF-1. Thus, an amino group is produced by the surface treatment process so that SDF-1 and collagen can be chemically attached to the scaffold through the amino group, whereby adhesion of SDF-1 and collagen to the scaffold can be ensured and cells can be stably attached to the scaffold.

This surface treatment process is as follows. First, 3-APTES is added to triple-distilled water in an amount of 5 to 15 vol % based on the volume of the triple-distilled water, and is thoroughly mixed with the triple-distilled water to obtain a mixture. Then, a calcium phosphate scaffold is immersed in the mixture at a temperature of 90 to 100° C. for 1 to 3 hours, and then washed thoroughly with distilled water and ultrasonic waves so that only reacted 3-APTES remains and unreacted 3-APTES is removed.

In this pretreatment process, the hydroxyl group (—OH) on the calcium phosphate scaffold reacts with the radical of 3-APTES so that 3-APTES bonds to the scaffold. As a result, as shown in FIG. 2, the hydroxyl group on the surface of the immersed calcium phosphate scaffold is replaced with an amino group (—NH₂).

(3) Preparation of Solution Containing Collagen and SDF-1

First solution: collagen is added to distilled water in an amount of 0.1 to 1.0 wt % based on the weight of the distilled water, and then 0.1-0.5 ml of acetic acid is added thereto and completely dissolved therein.

Second solution: 1-ethyl-3-(3-dimethylamino-propyl)carbodiimide (EDC) and N-hydroxysuccinimide (NHS) are mixed with triple-distilled water in an amount of 0.1 to 0.5 wt % based on the weight of the triple-distilled water.

The first solution and the second solution prepared as described above are mixed with each other at a weight of 1-2: 2-1 (preferably 1:1) and maintained for 10 hours (preferably 6 hours) to activate the carboxyl group. When the mixture solution of the first solution and the second solution reaches a weakly acidic pH of about 5.5-4.7, SDF-1 is added to the mixture solution in an amount of 0.1-1 wt % based on the total weight of the mixture solution and is completely dissolved therein, thereby preparing a solution containing collagen and SDF-1.

(4) Step of Coating Collagen and SDF-1 on Calcium Phosphate Scaffold

Collagen and SDF-1 can be simultaneously attached to the calcium phosphate scaffold, surface-treated with 3-APTES as described above, by chemical bonding and physical action, and thus the preparation process can be simplified.

Specifically, the calcium phosphate scaffold, surface-treated with 3-APTES in (2) above is immersed in the solution of collagen and SDF-1, prepared in (3) above, for about 4-6 hours. Then, the calcium phosphate scaffold having coated thereon collagen and SDF-1 is taken out, washed with triple-distilled water, and completely dried at room temperature.

In this process, as shown in FIG. 2, the amino group (—NH₂) and the carboxyl group (—COOH) of each of collagen and SDF-1 on the calcium phosphate scaffold, surface-treated with 3-APTES undergo dehydration condensation to remove water (H₂O) and to form a peptide bond (—CONH), and thus collagen and SDF-1 form chemical bonds to the calcium phosphate scaffold surface-treated with 3-APTES.

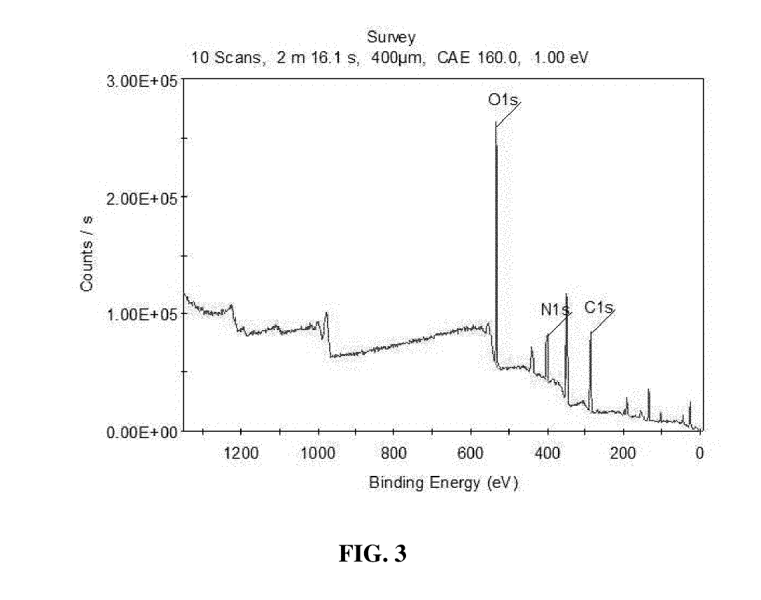

FIG. 3 shows the results of XPS (X-ray photoelectron spectroscopy) analysis of the surface of the scaffold according to the present invention. As shown therein, C1s detected at around about 300 eV, N1s detected at about 400 eV, and O1s detected at 500 eV or higher, are C, N and O, respectively, which result from a peptide bond. This indicates that collagen and SDF-1 were successfully coated on the surface of the calcium phosphate scaffold, surface-treated with 3-APTES, by chemical bonding.

In this case, non-chemically bonded SDF-1 can be physically attached to the surface of the calcium phosphate scaffold, surface-treated with 3-APTES, together with collagen fiber.

This scaffold according to the present invention absorbs cells and blood by a capillary force, and blood vessels and cells are introduced into the macropores of the scaffold. Then, integrin on the cells recognizes collagen, coated on the scaffold surface, as an extracellular matrix, thereby increasing the proliferation and differentiation rates of the cells.

In addition, the physically attached SDF-1 promotes the migration of cells to the scaffold to influence the early adhesion and proliferation of the cells, while the chemically bonded SDF-1 is involved in the continuous induction and introduction of stem cells into the scaffold. In addition, the macropores in the scaffold can increase the formation and occupation of autogenous bone.

Thus, the scaffold according to the present invention can increase bone regeneration rate and, at the same time, can greatly contribute to reducing the time required for restoration of bone defects. Accordingly, coating SDF-1 and collagen on a porous scaffold structure according to the present invention is advantageous in terms of bone regeneration.

1. A scaffold preparing method, comprising the steps of:
(S1) preparing a calcium phosphate scaffold including macropores having a size of 200 to 400 μm, and micro open pores having a size of 0.5 to 5 μm.
(S2) surface treating the scaffold prepared in step (S1) with 3-Aminopropyltriethoxysilane (3-APTES);
(S3) coating collagen and stromal cell-derivated factor-1 (SDF-1) on the scaffold surface-treated in step (S2) by immersing for 4 to 6 hours in a solution including the collagen and the SDF-1, wherein the solution has the SDF-1 in an amount of 0.1-1 wt % based on a total weight of the solution;
(S4) washing the scaffold coated in step (S3), and drying the washed scaffold at room temperature.

2. (canceled)

3. (canceled)

4. A scaffold prepared according to the method of claim 1, wherein the collagen and the SDF-1 are physically and chemically bonded to a surface of the scaffold.
